(19) United States
(12) Patent Application Publication
(10) Pub. No.: US 2025/0082252 A1
(43) Pub. Date: Mar. 13, 2025

(54) INPH PREDICTION METHOD AND DEVICE BASED ON THE NEW/OLD STIMULUS BCI PARADIGM

(71) Applicant: TIANJIN UNIVERSITY, Tianjin City (CN)

(72) Inventors: Xiuyun LIU, Tianjin City (CN); Dong MING, Tianjin City (CN); Congying HAO, Tianjin City (CN); Zihua ZHANG, Tianjin City (CN); Huijie YU, Tianjin City (CN); Yulin SUN, Tianjin City (CN); Xiaoyi WANG, Tianjin City (CN); Kai YU, Tianjin City (CN); Fang GUO, Tianjin City (CN); Kuo ZHANG, Tianjin City (CN); Meijun PANG, Tianjin City (CN)

(73) Assignee: TIANJIN UNIVERSITY, Tianjin City (CN)

(21) Appl. No.: 18/412,617

(22) Filed: Jan. 15, 2024

(30) Foreign Application Priority Data
Sep. 7, 2023 (CN) 2023111555900

(51) Int. Cl. A61B 5/369; G16H 30/40

(52) U.S. Cl. CPC A61B 5/369; G16H 30/40

(57) ABSTRACT

The current innovation pertains to the medical data processing domain and introduces a method for formulating an iNPH prediction model using a new/old stimulus BCI paradigm. The process involves conducting new/old stimulus BCI paradigm experiments on a target population both before and after the Lumbar Tap Test, obtaining electroencephalogram (EEG) signal data. After preprocessing the EEG signal data, the model extracts features related to new and old stimulus event-related potentials before and after the test, specifically focusing on P600 amplitude features. The iNPH prediction model is then trained based on these event-related potential features. This innovation facilitates quantifying cognitive function improvement, aiding physicians in prompt iNPH diagnosis and enabling timely and effective patient treatment.

### TECHNICAL FIELD

The present invention relates to the technical field of medical data processing, and in particular to an iNPH prediction method and apparatus based on the new/old stimulus BCI paradigm.

### BACKGROUND ART

Idiopathic Normal Pressure Hydrocephalus (iNPH) is a treatable but difficult to diagnose neurologic disorder. Patients exhibit the classic triad of symptoms, including gait disturbance, urinary incontinence, and cognitive impairment. The disease is most commonly seen in the elderly, with a significant increase in prevalence especially with age. Imaging findings show normal cerebrospinal fluid pressure and enlarged ventricles in patients with hydrocephalus. However, the disease is difficult to diagnose and differentiate due to the absence of an exact etiology; for example, in Europe and Japan, iNPH is considered an independently diagnosed disease, whereas in the United States it is categorized as a subtype of Alzheimer's disease. The similarity of the characteristics of multiple pathologies (e.g., Alzheimer's disease, dementia) leads to a situation where, if a misdiagnosis occurs, the patient will not be treated effectively, which can lead to exacerbation of the condition. Therefore, it is of great significance to explore more effective methods to aid in diagnosis, to identify potential iNPH patients, and to enable patients to receive treatment as early as possible.

Currently a positive pre- and post-Lumbar Tap Test is the gold standard for the diagnosis of iNPH. Various clinical tests that can be used to aid in the diagnosis of iNPH have also been outlined based on international and Japanese guidelines for iNPH, as well as a number of review articles. In addition to the triad of signs, radiologic and biochemical markers, these include the Tap Test (TT), Infusion test (IT), External Lumbar Drainage (ELD), and Intracranial Pressure (ICP) monitoring, where the Lumbar Tap Test (LTT) is a common clinical method to distinguish iNPH from other diseases. However, current guidelines do not include clear diagnostic parameters and thresholds for each test, so there is a lack of consistency in the approach and evaluation of each test in practice. Meanwhile, due to the diversity of clinical symptoms and the difficulty in differentiating from other neurological disorders, patients with iNPH often need to seek the help of multiple specialists, which is inefficient in diagnosis and leads to failure to receive timely and effective treatment.

Therefore, there is an urgent need for a method that can assist physicians in the rapid diagnosis of iNPH.

### SUMMARY

The present invention provides an iNPH prediction method and apparatus based on the new/old stimulus BCI paradigm to address the shortcoming that the prior art lacks a method that effectively assists a physician in rapidly diagnosing iNPH.

The present invention provides a method of constructing an iNPH prediction model based on a new/old stimulus BCI paradigm, comprising:
New/old stimulus BCI paradigm experiments were performed on the target population before Lumbar Tap Test to obtain pre-LTT new stimulus EEG signal data and pre-LTT old stimulus EEG signal data for the target population;
The same new/old stimulus BCI paradigm experiments were performed on the target population after Lumbar Tap Test to obtain post-LTT new stimulus EEG signal data and post-LTT old stimulus EEG signal data for the target population;
Pre-processing was performed on the pre-LTT new stimulus EEG signal data, pre-LTT old stimulus EEG signal data, post-LTT new stimulus EEG signal data and post-LTT old stimulus EEG signal data to obtain the pre-LTT new stimulus event-related potential features, the pre-LTT old stimulus event-related potential features, the post-LTT new stimulus event-related potential features and the post-LTT old stimulus event-related potential features, wherein the event-related potential features were the P600 amplitude features.

The iNPH prediction model was trained based on the pre-LTT new stimulus event-related potential features, pre-LTT old stimulus event-related potential features, post-LTT new stimulus event-related potential features and post-LTT old stimulus event-related potential features;

One of the target populations is iNPH patients.

A method of constructing an iNPH prediction model based on a new/old stimulus BCI paradigm according to the present invention, said performing a new/old stimulus BCI paradigm experiment on a target population comprising:
Configure EEG caps for the target population;
Presenting a number of images to a target population at predetermined time intervals, receiving image response information from the target population, wherein, in the new/old stimulus BCI paradigm experiment, the first presentation of an image is defined as a new stimulus and the non-first presentation of an image is defined as an old stimulus, and the target population is asked to memorize the presented images, and the image response information includes the response information that the target population thinks the image appears for the first time and the response information that the target population thinks the image does not appear for the first time;
While presenting an image to the target population and receiving image response information from the target population, EEG signal data of the target population is obtained by means of an EEG cap.

A method of constructing an iNPH prediction model based on a new/old stimulus BCI paradigm according to the present invention, said presenting a number of images to a target population according to a predetermined time interval, comprising:
A number of rounds of image stimulation are administered to the target population, each round of image stimulation comprising a black-and-white image stimulation and a color image stimulation, the black-and-white image stimulation and the color image stimulation comprising a number of image stimulations, wherein there is a break between each round of image stimulation and between the black-and-white image stimulation and the color image stimulation, and wherein the black-and-white image and the color image are derived from different stimulation libraries;
Images are presented to the target population each time in accordance with a predetermined stimulus maintenance duration, wherein one-half of the images appear only once, another one-half of the images are repeated once and there is a stimulus interval between the first image presentation and the second image presentation.

A method of constructing an iNPH prediction model based on the BCI paradigm for new/old stimuli according to the present invention, said configuring an EEG cap for a target population, specifically:
The target population was equipped with EEG caps, which used standard Ag/AgCl electrodes, with reference to the international 10-20 system of electrode placement, and the number of channels was 21 leads; the parameters of the EEG caps were set to a sampling rate of 1,000 Hz and a band-pass filtering of 0.1 to 200 Hz, and the acquisition process was performed with the top of the head of the target population as a reference, with the forehead grounded and the impedance between the scalp and the electrodes maintained at less than 10 KΩ.

A method of constructing an iNPH prediction model based on the new/old stimulus BCI paradigm according to the present invention, said preprocessing comprising band-pass filtering, re-referencing, independent component analysis, data segmentation, and baseline correction; wherein
Band-pass filtering is specified as: the filtering of EEG signal data by a band-pass filter with a filter range of 0.5 to 20 Hz;
Re-referencing is specified as: the EEG signal data were re-referenced to the average of the left mastoid and right mastoid as a reference;
Independent component analysis is specified as: the identification of EOG and EMG components associated with artifacts in order to remove artifacts from the EEG signal data;
The data segmentation is specified as: defining the moment of appearance of the image stimulus as the zero moment, in the EEG signal data, the data is segmented and intercepted according to the first predetermined time window;
The baseline calibration is specified as: using the EEG signal data segments of the second predetermined time window as a baseline, subtracting the EEG signal data segments of the first predetermined time window from the average value of the EEG signal data segments of the baseline to eliminate the drift of the EEG signal data relative to the baseline.

A method of constructing an iNPH prediction model based on the new/old stimulus BCI paradigm according to the present invention, said pre-processing thepre-LTT new stimulus EEG signal data, thepre-LTT old stimulus EEG signal data, the post-LTT new stimulus EEG signal data, and the post-LTT old stimulus EEG signal data to obtain thepre-LTT new stimulus event-related potential features, thepre-LTT old stimulus event-related potential features, the post-LTT post-LTT new stimulus event-related potential features and post-LTT old stimulus event-related potential features, including:

Based on the preprocessed EEG signal data, the event-related potential features were obtained by averaging the EEG signal data of each trial separately at each electrode by overlaying the calculation.

According to a method of constructing an iNPH prediction model based on an new/old stimulus BCI paradigm provided by the present invention, the event-related potential features of the target population are expressed in the form of: a new stimulus event-related potential features before Lumbar Tap Test, an old stimulus event-related potential features before Lumbar Tap Test, a new stimulus event-related potential features after Lumbar Tap Test, and a new stimulus event-related potential features after Lumbar Tap Test, and the old stimulus event-related potential features after Lumbar Tap Test reflecting that the iNPH population undergoing Lumbar Tap Test the absence of the old-new effect before Lumbar Tap Test and the appearance of the old-new effect after Lumbar Tap Test;
where the old-new effect indicates that the P600 amplitude feature under the old stimulus has a more positive potential than the P600 amplitude feature under the new stimulus within a certain time frame;

Said iNPH prediction model is trained to obtain an iNPH prediction model based on a pre-LTT new stimulus event-related potential feature, a pre-LTT old stimulus event-related potential feature, a post-LTT new stimulus event-related potential feature, and a post-LTT old stimulus event-related potential feature, as follows:

The iNPH prediction model was trained to obtain the iNPH prediction model based on the feature changes reflected in the pre-LTT new stimulus event-related potential features, pre-LTT old stimulus event-related potential features, post-LTT new stimulus event-related potential features, and post-LTT old stimulus event-related potential features of different target populations.

The present invention also provides an apparatus for constructing an iNPH prediction model based on a new/old stimulus BCI paradigm, comprising:

A data receiving module, said data receiving module being configured to: receive pre-LTT new stimulation EEG signal data, pre-LTT old stimulation EEG signal data, post-LTT new stimulation EEG signal data, and post-LTT old stimulation EEG signal data of the target population, wherein said pre-LTT new stimulation EEG signal data and pre-LTT old stimulation EEG signal data, and post-LTT new stimulation EEG signal data and post-LTT old stimulated EEG signal data, and post-LTT new stimulated EEG signal data and post-LTT old stimulated EEG signal data were obtained through the BCI paradigm experiments in which the target population received new and old stimulated BCI paradigm experiments before and after lumbar puncture and lumbar Tap Test, respectively;
A data processing module, said data processing module being configured to: pre-process the pre-LTT new stimulus EEG signal data, the pre-LTT old stimulus EEG signal data, the post-LTT new stimulus EEG signal data and the post-LTT old stimulus EEG signal data received by said data receiving module, to obtain the pre-LTT new stimulus event-related potential feature, the pre-LTT old stimulus event-related potential feature, the post-LTT new stimulus event-related potential features, old stimulus event-related potential features, new stimulus event-related potential features, new stimulus event-related potential features and old stimulus event-related potential features after LTT, of which, the event-related potential features are P600 amplitude features;
A model training module, said model training module being configured to: train to obtain an iNPH prediction model based on the pre-LTT new stimulus event related potential features, the pre-LTT old stimulus event related potential features, the post-LTT new stimulus event related potential features and the post-LTT old stimulus event related potential features obtained by said data processing module.

The present invention also provides an iNPH prediction device based on the new/old stimulus BCI paradigm, comprising:

A data acquisition module, said data acquisition module being configured to: acquire pre-LTT new stimulus EEG signal data and pre-LTT old stimulus EEG signal data obtained from an experiment in which the tester received new/old stimulus BCI paradigms prior to performing a lumbar Tap Test, and to acquire post-LTT new stimulus EEG signal data and post-LTT old stimulus EEG signal data obtained from an experiment in which the tester received new/old stimulus BCI paradigms subsequent to performing a lumbar Tap Test Data;
A probabilistic prediction module, said probabilistic prediction module being configured to: input the pre-LTT new stimulus EEG signal data, the pre-LTT old stimulus EEG signal data, the post-LTT new stimulus EEG signal data, and the post-LTT old stimulus EEG signal data of the tester obtained by said data acquisition module into the iNPH obtained by the method of constructing an iNPH prediction model based on new/old stimulus BCI paradigms as described in any one of the foregoing prediction model to obtain an iNPH prediction result;
Wherein the tester is a patient with suspected iNPH or a patient to be ruled out with iNPH, preferably said tester has a triad of manifestations of gait disturbance, urinary incontinence and cognitive impairment.

The present invention also provides an electronic device comprising a processor and a memory storing a computer program, characterized in that said processor implements the following steps when executing said program:
Obtain pre-LTT new stimulus EEG signal data and pre-LTT old stimulus EEG signal data obtained from the BCI paradigm experiment in which the tester received new/old stimuli before Lumbar Tap Test, as well as obtain post-LTT new stimulus EEG signal data and post-LTT old stimulus EEG signal data obtained from the BCI paradigm experiment in which the tester received new/old stimuli after Lumbar Tap Test;
The pre-LTT new stimulus EEG signal data, the pre-LTT old stimulus EEG signal data, the post-LTT new stimulus EEG signal data, and the post-LTT old stimulus EEG signal data of the tester are inputted into the iNPH prediction model obtained by the method of constructing an iNPH prediction model based on the new/old stimulus BCI paradigm described in any one of the foregoing, and the iNPH prediction results are obtained;
Wherein the tester is a patient with suspected iNPH or a patient to be ruled out with iNPH, preferably said tester has a triad of manifestations of gait disturbance, urinary incontinence and cognitive impairment.

The present invention also provides a brain-computer interface system for predicting iNPH, comprising:
An EEG cap for being worn on the head of a tester to obtain EEG signals from said tester;
An EEG acquisition device configured to be coupled to the electrodes of said EEG cap to acquire EEG signals of said tester acquired by said EEG cap;
A computer comprising a communication module, a memory, a processor, and a computer program stored on said memory and runnable on said processor, wherein said computer is configured to communicate with said electroencephalographic acquisition device to obtain an electroencephalographic signal from said tester, and wherein said processor is configured to implement the following steps in executing said computer program:

Obtain pre-LTT new stimulus EEG signal data and pre-LTT old stimulus EEG signal data obtained from the BCI paradigm experiment in which the tester received new/old stimuli prior to performing a lumbar Tap Test, as well as obtain post-LTT new stimulus EEG signal data and post-LTT old stimulus EEG signal data obtained from the BCI paradigm experiment in which the tester received new/old stimuli after performing a lumbar Tap Test;
Preprocessing was performed on the pre-LTT new stimulus EEG signal data, the pre-LTT old stimulus EEG signal data, the post-LTT new stimulus EEG signal data, and the post-LTT old stimulus EEG signal data;
The preprocessed pre-LTT new stimulus EEG signal data, the pre-LTT old stimulus EEG signal data, the post-LTT new stimulus EEG signal data, and the post-LTT old stimulus EEG signal data are inputted into an iNPH prediction model obtained by the method of constructing an iNPH prediction model based on the new/old stimulus BCI paradigm described in any one of the foregoing claims, and a prediction result as to whether or not said tester is a patient with iNPH is obtained;
wherein said computer program comprises an EEG analysis program (e.g., EEG processing toolbox EEGLAB) for pre-processing EEG signals acquired by said EEG acquisition device;
Wherein the tester is a patient with suspected iNPH or a patient to be ruled out with iNPH, preferably said tester has a triad of manifestations of gait disturbance, urinary incontinence and cognitive impairment.

The present invention provides an iNPH prediction method and apparatus based on the new/old stimulus BCI paradigm, in which an Electroencephalography (EEG)-based new/old stimulus BCI paradigm experiment is carried out on a target population before and after Lumbar Tap Test, and an Event-Related Potentials (ERP) feature change is captured during short-term memory images of the target population. ERP features (ERPs) of the target population during short-term memory images, and train the iNPH prediction model based on the new stimulus ERPs before lumbar Tap Test, old stimulus ERPs before lumbar Tap Test, new stimulus ERPs after lumbar Tap Test, and old stimulus ERPs after lumbar Tap Test (P600 amplitude features), to quantify the degree of cognitive function improvement of the target population. The iNPH prediction model can assist doctors to diagnose iNPH quickly, so that patients can get effective treatment in time.

### BRIEF DESCRIPTION OF THE DRAWINGS

In order to more clearly illustrate the technical solutions in the present invention, the following will make a brief introduction to the accompanying drawings that need to be used in the description of the embodiments or prior art, and it will be obvious that the accompanying drawings in the following description are some of the embodiments of the present invention, and for the person of ordinary skill in the field, other accompanying drawings can be obtained based on these drawings without putting in creative labor.

FIG. 1 shows one of the process schematic diagrams of a method for constructing an iNPH prediction model based on a new/old stimulus BCI paradigm provided by the present invention.

FIG. 2 shows the EEG cap 21-lead position map.

FIG. 3 shows one of the schematic diagrams of the experimental flow of the new/old stimulus BCI paradigms.

FIG. 4 shows the time-domain waveforms of the Pz leads in a healthy population stimulated with black-and-white images.

FIG. 5 shows the time-domain waveforms of the Pz leads in a healthy population stimulated with color images.

FIG. 6 (a) shows the time-domain waveforms of the Pz leads in the iNPH population stimulated with black-and-white images before Lumbar Tap Test.

FIG. 6 (b) shows the time-domain waveforms of Pz leads in the iNPH population stimulated with black-and-white images 24h after Lumbar Tap Test.

FIG. 6 (c) shows the time-domain waveforms of the Pz leads in the iNPH population under black-and-white image stimulation 72h after Lumbar Tap Test.

FIG. 7 (a) shows the time-domain waveforms of the Pz leads in the iNPH population under color image stimulation before Lumbar Tap Test.

FIG. 7 (b) shows the time-domain waveforms of the Pz leads in the iNPH population under color image stimulation 24h after Lumbar Tap Test.

FIG. 7 (c) shows the time-domain waveforms of the Pz leads in the iNPH population under color image stimulation 72h after Lumbar Tap Test.

FIG. 8 shows a schematic structure of an iNPH prediction system based on the new/old stimulus BCI paradigm provided by the present invention.

FIG. 9 shows a schematic diagram of the structure of an electronic device provided by the present invention.

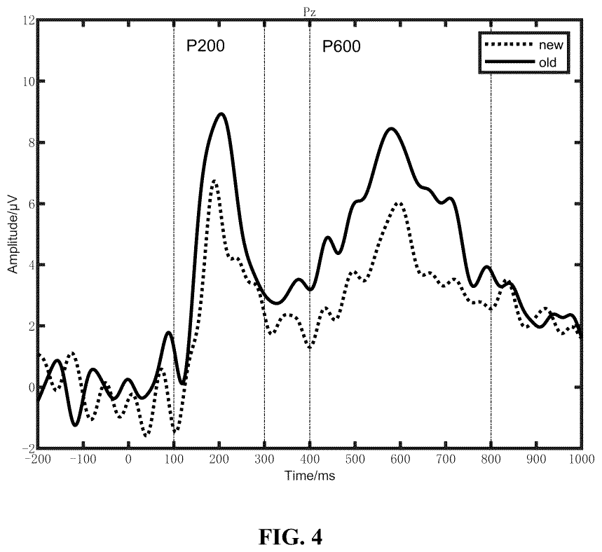

Where, in FIG. 4-FIG. 7, new denotes new stimulus, old denotes old stimulus.

### DETAILED DESCRIPTION OF THE EMBODIMENTS

In order to make the objects, technical solutions and advantages of the present invention clearer, the technical solutions in the present invention will be described clearly and completely in the following in combination with the accompanying drawings in the present invention, and it is obvious that the described embodiments are part of the embodiments of the present invention, not all of the embodiments, and they should not be construed as limitations of the present invention. Based on the embodiments in the present invention, all other embodiments obtained by a person of ordinary skill in the art without making creative labor are within the scope of protection of the present invention. In the description of the present invention, it is to be understood that the terms used are used for descriptive purposes only and are not to be construed as indicating or implying relative importance.

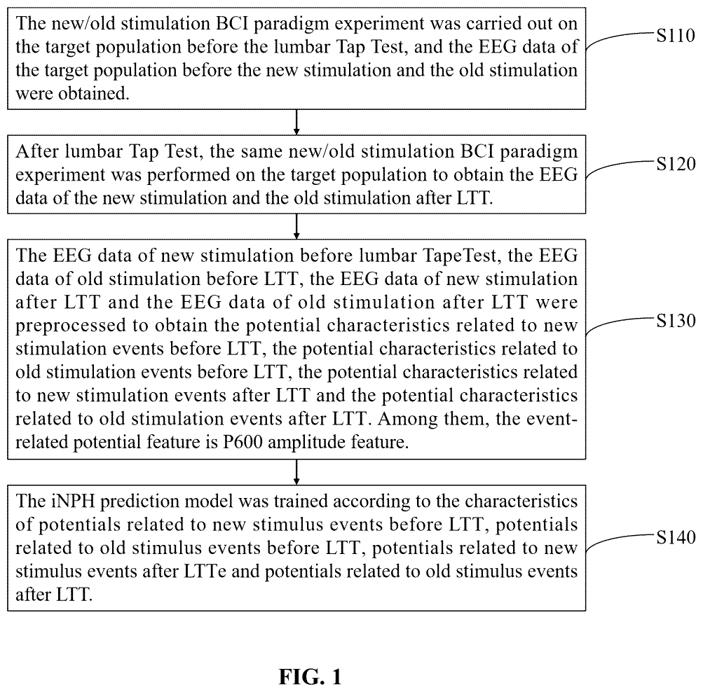

The iNPH prediction method and apparatus based on the new/old stimulus BCI paradigm provided by the present invention are described below in conjunction with FIG. 1-FIG. 9.

FIG. 1 is one of the flow schematic diagrams of a method of constructing an iNPH prediction model based on a new/old stimulus BCI paradigm provided by the present invention. Referring to FIG. 1, a method of constructing an iNPH prediction model based on a new/old stimulus BCI paradigm provided by the present invention may comprise:

Step S110: Before the target population is subjected to Lumbar Tap Test, the target population is subjected to a new/old stimulation BCI paradigm experiment, and the pre-LTT new stimulation EEG signal data and the pre-LTT old stimulation EEG signal data of the target population are obtained;

Step S120: After the target population is subjected to Lumbar Tap Test, the same new/old stimulation BCI paradigm experiment is performed on the target population to obtain the post-LTT new stimulation EEG signal data and the post-LTT old stimulation EEG signal data of the target population;

Step S130: preprocessing the pre-LTT new stimulus EEG signal data, the pre-LTT old stimulus EEG signal data, the post-LTT new stimulus EEG signal data, and the post-LTT old stimulus EEG signal data to obtain the pre-LTT new stimulus event-related potential feature, the pre-LTT old stimulus event-related potential feature, the post-LTT new stimulus event-related potential feature, and the post-LTT old stimulus event-related potential feature, wherein The event-related potential feature is the P600 amplitude feature, indicating the amplitude feature near the 600 ms after the stimulus occurrence;

Step S140: The iNPH prediction model is trained to obtain the iNPH prediction model based on the pre-LTT new stimulus event-related potential feature, the pre-LTT old stimulus event-related potential feature, the post-LTT new stimulus event-related potential feature, and the post-LTT old stimulus event-related potential feature.

It is important to note that Brain Computer Interface (BCI) is a technique for direct communication between the human brain and an output device, due to the advantages of Electroencephalography (EEG) which is non-invasive, easy to use and low cost.

It should be noted that the target population is iNPH patients.

It should be noted that this embodiment performs the same new/old stimulation BCI paradigm experiments on the target population before lumbar Tap Test, 24h after lumbar Tap Test, and 72h after lumbar Tap Test, which makes the experimental data more convincing.

In one embodiment, said experiments with new/old stimulus BCI paradigms on a target population comprise:
Configure EEG caps for the target population;
Presenting a number of images to a target population at predetermined time intervals, receiving image response information from the target population, wherein, in the new/old stimulus BCI paradigm experiment, the first presentation of an image is defined as a new stimulus and the non-first presentation of an image is defined as an old stimulus, and the target population is asked to memorize the presented images, and the image response information includes response information in which the target population perceives that the image is first time appearing and response information in which the target population perceives that the image non-first appearance response information;
While presenting an image to the target population and receiving image response information from the target population, EEG signal data of the target population is obtained by means of an EEG cap.

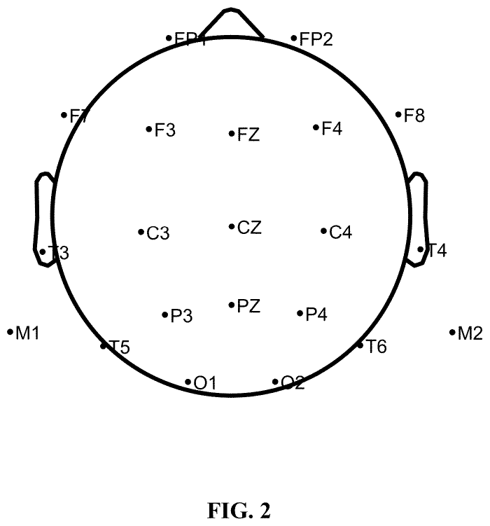

In one embodiment, referring to FIG. 2, an EEG cap is configured for the target population, specifically: the EEG cap adopts standard Ag/AgCl electrodes, the electrodes are placed with reference to the international 10-20 system, and the number of channels is 21 leads, the parameters of the EEG cap are set to a sampling rate of 1,000 Hz and a band-pass filtering from 0.1 to 200 Hz, and at the same time, the 50 Hz trap is used to filter out the interference of the work frequency, and the acquisition process takes the target population the top of the head of the target population was used as the reference, the forehead was grounded, and the impedance between the scalp and the electrodes was kept below 10KΩ. During the BCI paradigm experiments with new/old stimuli, it is necessary to ask the target population to remain as still as possible, avoiding random eye movements and subtle movements not related to the task, so as to ensure the reliability of the collected EEG data.

Specifically, this embodiment employs an EEG acquisition device, Neuroscan amplifier to be connected to the EEG cap to enable the acquisition of EEG signal data.

In one embodiment, said presenting a number of images to a target population at predetermined time intervals comprises:
A number of rounds of image stimulation are administered to the target population, each round of image stimulation comprising a black-and-white image stimulation and a color image stimulation, wherein a rest period is provided between each round of image stimulation and between the black-and-white image stimulation and the color image stimulation, and the black-and-white image and the color image are derived from different stimulation libraries;
Images are presented to the target population each time in accordance with a predetermined stimulus maintenance duration, wherein one-half of the images appear only once, another one-half of the images are repeated once and there is a stimulus interval between the first image presentation and the second image presentation.

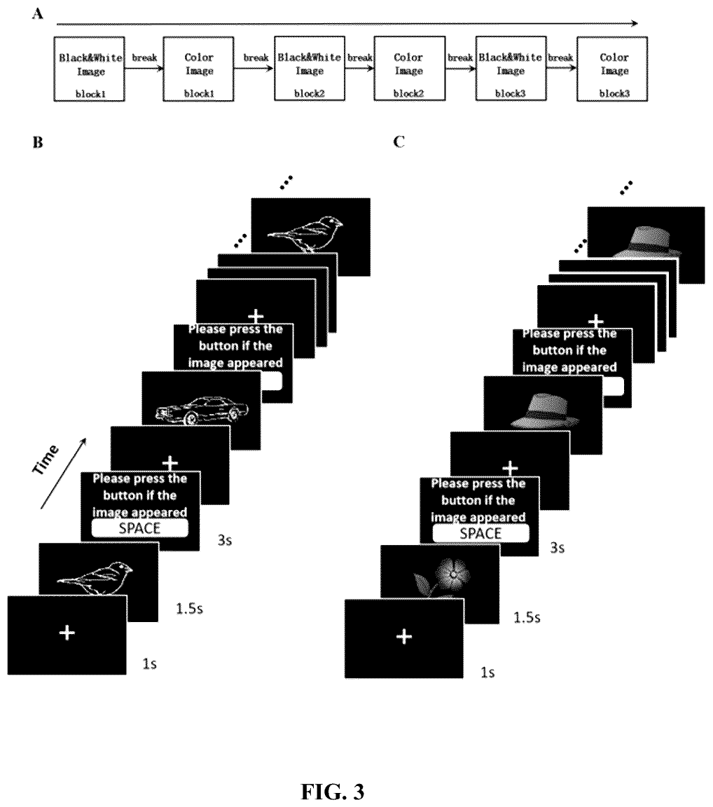

Specifically, with reference to FIG. 3, the present embodiment performs the same new/old stimulus BCI paradigm experiment (which can be realized by presenting a series of image stimuli on a screen) on the target population before Lumbar Tap Test, 24h after Lumbar Tap Test, and 72h after Lumbar Tap Test, respectively, and a new/old stimulus BCI paradigm experiment consists of three rounds of image stimuli (six blocks), and each round of image stimuli consists of one round of Each round of image stimulation included one round of black-and-white image stimulation and one round of color image stimulation (one round consisted of two blocks), each round of black-and-white image stimulation and each round of color image stimulation consisted of a number of image stimuli (each block consisted of 80 image stimuli), one-half of the images appeared once only, and the other one-half of the images were repeated once with a stimulus interval of 40 to 70 s (10 to 13 trials) between the first and the second image presentation. Each new/old stimulus BCI paradigm experiment required six rounds of image stimulation for the target population, three rounds of black-and-white image stimulation plus three rounds of color image stimulation, and the black-and-white image stimulation and the color image stimulation were interspersed, and there was a rest period between each round of image stimulation as well as between black-and-white image stimulation and color image stimulation (5-10 min in this embodiment). In each trial, a white cross was presented in the center of the screen for Is to remind the target population to pay attention, and then an image was randomly presented on the screen (the image was a common object in daily life, such as fruits, animals, furniture, etc.), and the preset duration of each image stimulus in black-and-white image stimulation or color image stimulation was 1.5s. During the experiment, the target population was asked to memorize the objects presented in the images as much as possible, and at the end of each image stimulus, a selection interface could be presented on the screen, and the target population was asked to determine whether the image had been presented before and to respond to it (pressing the space bar on the keyboard if it was, or not if it was not a previously presented image), with the response limited to 3s, so that the target population's response information to the images could be obtained. In order to keep the target population motivated for the next experiments and to ensure the smooth running of the experiments, at the end of each block, the target population can be sent the correctness of their keystrokes.

Based on the EEG signal data generated by the target population in response to the image memorization during the new/old stimulus BCI paradigm experiments, a Pz-lead time-domain waveform map (also known as an ERP waveform map) can be produced to observe the feature changes. In the new/old stimulus BCI paradigm experiment conducted on the target population in this embodiment, the target population is in a passive state, which only needs to memorize the stimulus image and provide the stimulus image response information, and does not need to carry out complex understanding, which excludes the influence on the experimental results by the subjective factors of the target population, such as the cultural level of the target population; furthermore, this experimental method is low-cost and short time-consuming, and it is capable of assisting a physician in rapidly diagnosing iNPH, and effectively reduce medical cost and medical burden.

In one embodiment, said preprocessing comprises bandpass filtering, re-referencing, independent component analysis, data segmentation, and baseline correction; wherein the Band-pass filtering is specified as: the EEG signal data is filtered by a Butterworth band-pass filter with a filtering range of 0.5-20 Hz to remove the very low frequency and very high frequency interferences in the EEG signal;

Re-referencing is specified as: the EEG signal data were re-referenced to the average of the left mastoid and right mastoid as a reference;

Independent component analysis is specified as: the identification of EOG and EMG components associated with artifacts by EEGLAB in order to remove artifacts from the EEG signal data;

The data segmentation is specified as: the moment at which the image stimulus appears is defined as the zero moment, and in the EEG signal data, the data is segmented and intercepted in accordance with a first predetermined time window (in this embodiment, it is 0.2s before the zero moment to 1s after the zero moment);

The baseline calibration is specified as: using the EEG signal data segment of the predetermined second time window (0.2s before the zero moment in this embodiment) as a baseline, subtracting the average of the EEG signal data segment of the baseline from the EEG signal data segment of the first predetermined time window, so as to eliminate the drift of the EEG signal data relative to the baseline.

Specifically, this embodiment employs an EEG processing toolbox (EEGLAB) developed based on MATLAB to preprocess the pre-LTT new stimulation EEG signal data, the pre-LTT old stimulation EEG signal data, the post-LTT new stimulation EEG signal data, and the post-LTT old stimulation EEG signal data, to ensure that the precision of the processed EEG signal data is higher, which helps to improve the accuracy of the model prediction results.

In one embodiment, said pre-processing of pre-LTT new stimulation EEG signal data,pre-LTT old stimulation EEG signal data, post-LTT new stimulation EEG signal data and post-LTT old stimulation EEG signal data to obtain a pre-LTT new stimulation event-related potential features, a pre-LTT old stimulation event-related potential features, a post-LTT new stimulation event-related potential features and a post-LTT old stimulation event-related potential features, including:

Based on the preprocessed EEG signal data, the event-related potential features were obtained by averaging the EEG signal data of each trial separately at each electrode by overlaying the calculation.

The purpose of obtaining event-related potential features by superimposed averaging calculation is, on the one hand, because superimposed averaging of single trials can yield highly reproducible ERP waveforms for each stimulus type, and on the other hand, for each trial, the ERP waveforms are the same, and the noise is completely independent of the time-locked event, and the effect of artifacts can be further excluded by superimposed averaging to ensure the accuracy of the data. To test the ERP effect, the ERP of the first and second presentation of the image can be calculated separately.

It is important to note that the event-related potential features of different target populations are expressed in different ways, where the

Healthy people do not need to undergo lumbar Tap Test, healthy people in the normal state to receive the new/old stimulus BCI paradigm experiments, the event-related potential features of the manifestation of the new stimulus event-related potential features and the old stimulus event-related potential features reflect that the healthy people in the normal state has the old-new effect;

The event-related potential features of the iNPH population were expressed in the form of: pre-LTT new stimulus event-related potential features, pre-LTT old stimulus event-related potential features, post-LTT new stimulus event-related potential features, and post-LTT old stimulus event-related potential features reflecting the absence of the old-new effect before lumbar Tap Test and the appearance of the old-new effect after lumbar Tap Test in the iNPH population;

Wherein, the old-new effect indicates that the P600 amplitude feature under the old stimulus has a more positive potential than the P600 amplitude feature under the new stimulus within a certain time range, for example, it may be that the P600 amplitude feature under the old stimulus is higher and above a certain predetermined range than the P600 amplitude feature under the new stimulus, or it may be possible to set up a definition of having a more positive potential according to the actual experimental situation.

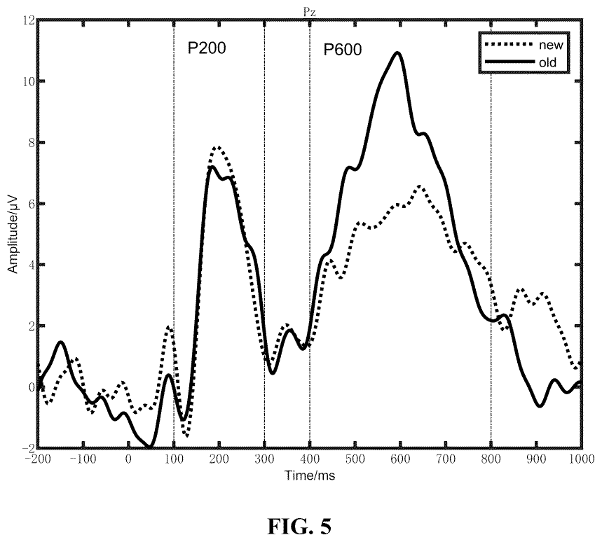

Specifically, for healthy population subjects, the results of the existing experiments are shown in FIG. 4 and FIG. 5, where the dashed line indicates the ERP waveform under the new stimulus (new, the first presentation of the image) and the solid line indicates the ERP waveform under the old stimulus (old, the second presentation of the image). In black-and-white image stimuli, comparing the ERP evoked by the new stimulus with that evoked by the old stimulus, a unique effect can be observed under the parietal leads (P3, P4, Pz), i.e., the P600 amplitude feature under the old stimulus has a more positive potential than that under the new stimulus for 400-800 ms, which is referred to as the old-new effect. Similarly the phenomenon can be observed in color image stimuli. Meanwhile, regardless of new or old stimuli, the P200 amplitude feature can be clearly observed around 200 ms, which reflects that the subjects' attention remained focused during the experiment. That is, the pre-LTT new stimulus event-related potential feature and the pre-LTT old stimulus event-related potential feature, which reflect that the target population has already had the old-new effect before lumbar Tap Test, can be used to predict that the target population is a healthy population/non-iNPH population.

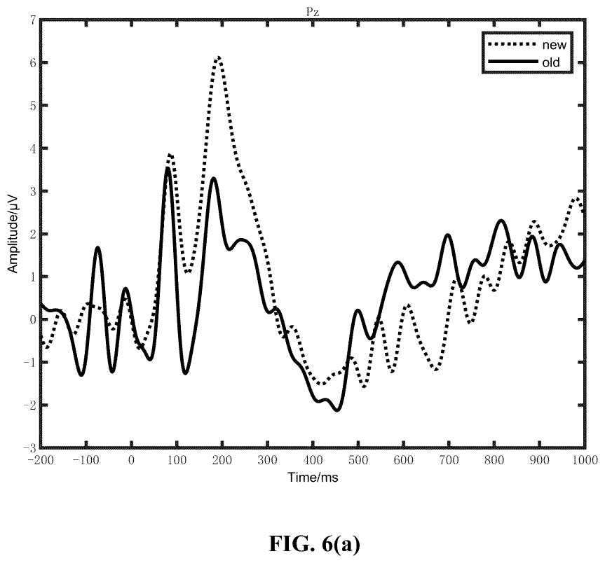

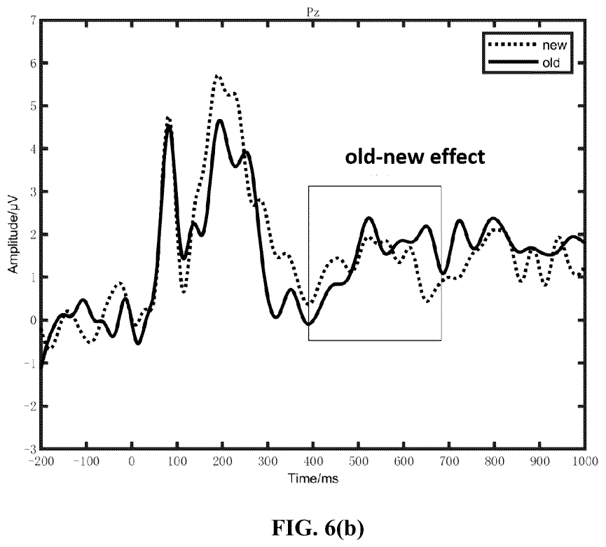

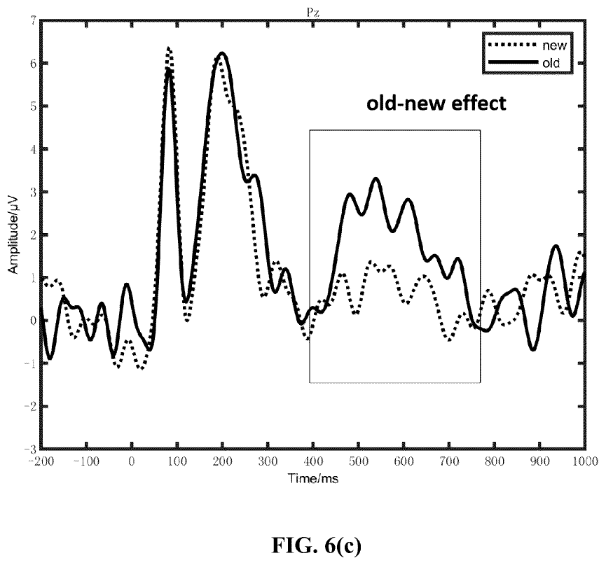

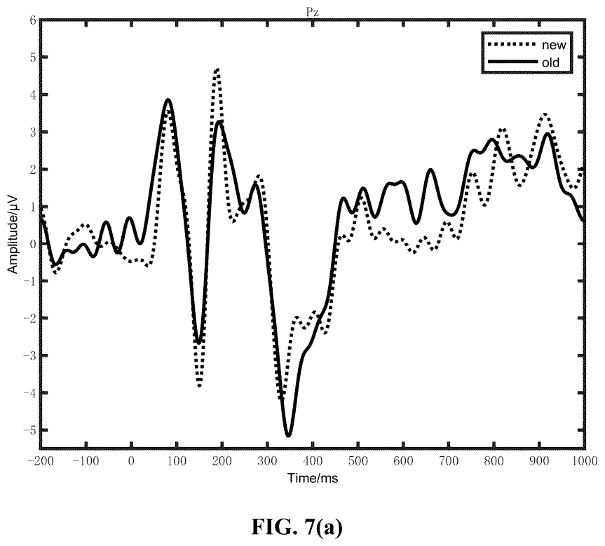

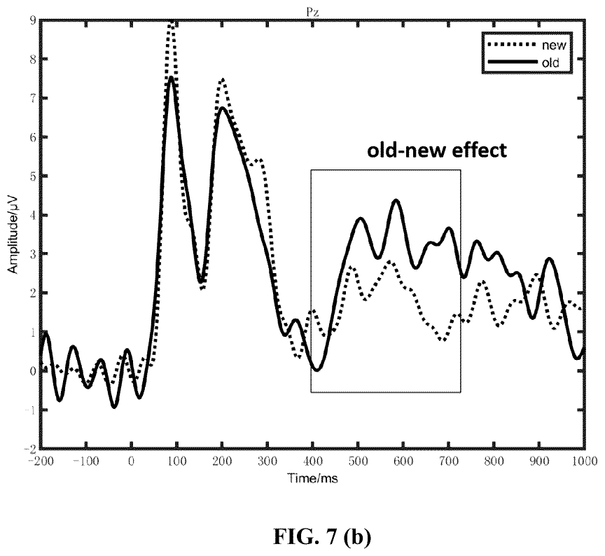

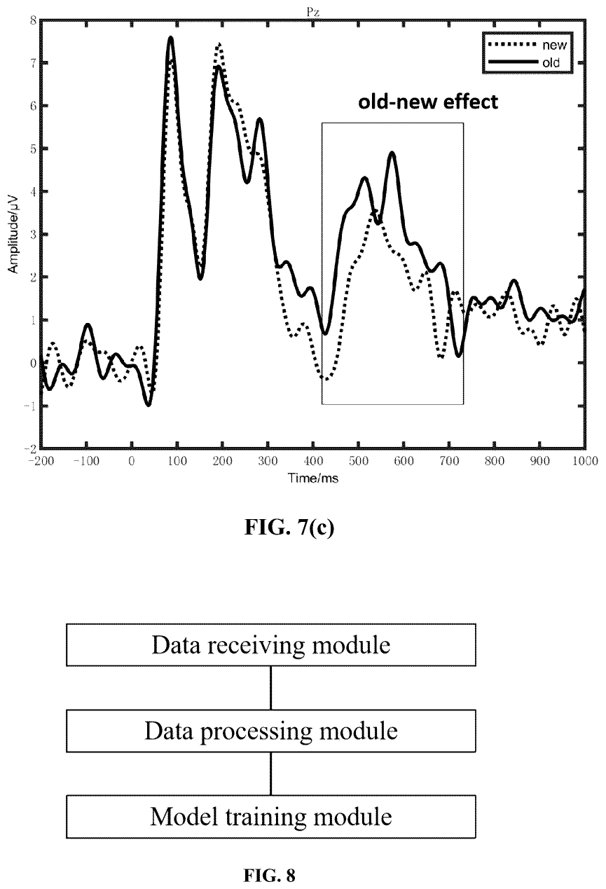

As for the iNPH population and other populations with age-related degenerative diseases such as Alzheimer's and dementia, the old-new effect itself does not exist, there is no difference in the characteristics of the P600 amplitude evoked by either the new stimulus or the old stimulus. However, after the iNPH population underwent lumbar Tap Test, the old-new effect appeared in their ERP waveform maps, indicating that the cognitive function of iNPH was improved, which is in line with the characteristics of the iNPH population, see the experimental results in FIG. 6 (a)-(c) as well as FIG. 7 (a)-(c), whereby the old-new effect did not exist in the iNPH population either in the black-and-white image stimulus or in the color image stimulus before the lumbar Tap Test was carried out, and the old-new effect appeared in the old-new effect appeared after the lumbar Tap Test, and the old-new effect was especially obvious after 72h of lumbar Tap Test. This indicates that the pre-LTT new stimulus event-related potentials, pre-LTT old stimulus event-related potentials, post-LTT new stimulus event-related potentials, and post-LTT old stimulus event-related potentials, which reflect that the target population does not have the old-new effect prior to lumbar Tap Test, but has the old-new effect after lumbar Tap Test, are able to be used for the prediction of the target population as the iNPH population.

Therefore, the iNPH prediction model can be trained to obtain the iNPH prediction model based on the change characteristics of the new stimulus event-related potential feature before lumbar Tap Test, the old stimulus event-related potential feature before lumbar Tap Test, the new stimulus event-related potential feature after lumbar Tap Test and the old stimulus event-related potential feature after lumbar Tap Test, so that the iNPH prediction model can be used to predict whether a tester is a patient with iNPH based on whether the data to be measured reflect the presence of the old-new effect before lumbar Tap Test, and whether there is an old-new effect from none to none before and after lumbar Tap Test, in order to assist doctors in judging the tester. The iNPH prediction model can be used to predict whether the tester is a patient with iNPH based on whether the data to be measured reflect the presence of old-new effect before the Lumbar Tap Test, and whether the old-new effect occurs before and after the Lumbar Tap Test, from the absence of the old-new effect to the presence of the old-new effect, in order to assist the doctor's judgment on the test.

It should be noted that the framework of the iNPH prediction model may use any of the compliant modeling frameworks in the prior art, such as a classification modeling framework, etc., which are not limited herein.

After training to obtain the iNPH prediction model, the model can be utilized for iNPH prediction by first conducting the new/old stimulus BCI paradigm experiments on the tester before the tester performs lumbar Tap Test, and obtaining the tester's pre-LTT new stimulus EEG signal data and the pre-LTT old stimulus EEG signal data; and then, after the tester performs lumbar Tap Test, conducting the same new/old stimulus BCI paradigm experiments on the tester, and obtain the post-LTT new stimulation EEG signal data and post-LTT old stimulation EEG signal data of the tester; and then, based on the pre-LTT new stimulation EEG signal data, the pre-LTT old stimulation EEG signal data, the post-LTT new stimulation EEG signal data, and the post-LTT old stimulation EEG signal data of the tester, the iNPH prediction obtained through the above method of constructing an iNPH prediction model based on the new/old stimulation BCI paradigm; wherein the testers include a patient with suspected iNPH, a patient to be ruled out as having iNPH, or a patient having a triadic manifestation of gait disorder, urinary incontinence, and cognitive impairment.

The present invention designs an new/old stimulus BCI paradigm based on BCI technology, which is sensitive to changes in the recognition memory process of iNPH patients. The P600 amplitude characteristics of the tester before and after LTT are induced by image stimulation and compared to the presence of old-new effect in the memory process, thus realizing a rapid and accurate diagnosis of iNPH and determining whether to perform subsequent bypass surgery. This technique overcomes the limitations of traditional iNPH diagnosis and has a wide range of medical applications, and at the same time can provide innovative ideas for the diagnosis of other diseases, which is expected to bring considerable social and economic benefits.

The present invention provides an iNPH prediction method and apparatus based on the new/old stimulus BCI paradigm, in which an Electroencephalography (EEG)-based new/old stimulus BCI paradigm experiment is carried out on a target population before and after Lumbar Tap Test, and an Event-Related Potentials (ERP) feature change during short-term memory images is captured in the target population. Potentials (ERP) characteristics of the target population during short-term memory images, and train the iNPH prediction model based on the new stimulus ERP characteristics before lumbar Tap Test, old stimulus ERP characteristics before lumbar Tap Test, new stimulus ERP characteristics after lumbar Tap Test, and old stimulus ERP characteristics after lumbar Tap Test (P600 amplitude characteristics), to quantify the degree of cognitive improvement of the target population. The iNPH prediction model can assist doctors to quickly diagnose whether a test subject is an iNPH patient, improve diagnostic efficiency, and enable patients to receive timely and effective treatment.

The construction apparatus for an iNPH prediction model based on a new/old stimulus BCI paradigm provided by the present invention is described below, and the construction apparatus for an iNPH prediction model based on a new/old stimulus BCI paradigm described below may be cross-referenced in correspondence to the construction method for an iNPH prediction model based on a new/old stimulus BCI paradigm described above.

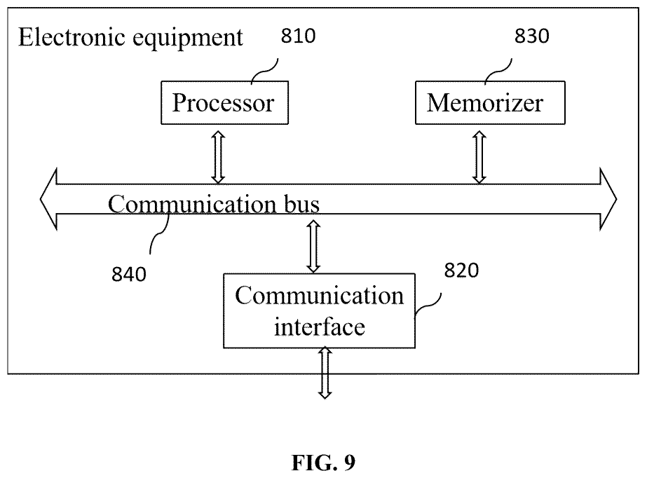

Referring to FIG. 8, an apparatus for constructing an iNPH prediction model based on a new/old stimulus BCI paradigm provided by the present invention may comprise:

A data receiving module, said data receiving module being configured to: receive pre-LTT new stimulation EEG signal data, pre-LTT old stimulation EEG signal data, post-LTT new stimulation EEG signal data, and post-LTT old stimulation EEG signal data of the target population, wherein said pre-LTT new stimulation EEG signal data and pre-LTT old stimulation EEG signal data, and post-LTT new stimulation EEG signal data and post-LTT old stimulated EEG signal data, and post-LTT new stimulated EEG signal data and post-LTT old stimulated EEG signal data were obtained by the target population receiving new/old stimulated BCI paradigm experiments before and after lumbar Tap Test, respectively;

A data processing module, said data processing module being configured to: pre-process the pre-LTT new stimulus EEG signal data, the pre-LTT old stimulus EEG signal data, the post-LTT new stimulus EEG signal data and the post-LTT old stimulus EEG signal data received by said data receiving module, to obtain thepre-LTT new stimulus event-related potential feature, the pre-LTT old stimulus event-related potential feature, the post-LTT new stimulus event-related potential features, old stimulus event-related potential features, new stimulus event-related potential features, new stimulus event-related potential features and old stimulus event-related potential features after lumbar Tap Test, of which, the event-related potential features are P600 amplitude features;

A model training module, said model training module being configured to: train to obtain an iNPH prediction model based on the pre-LTT new stimulus event related potential features, the pre-LTT old stimulus event related potential features, the post-LTT new stimulus event related potential features and the post-LTT old stimulus event related potential features obtained by said data processing module.

The present invention also provides an iNPH prediction device based on the new/old stimulus BCI paradigm, which may comprise:

A data acquisition module, said data acquisition module being configured to: acquire pre-LTT new stimulus EEG signal data and pre-LTT old stimulus EEG signal data obtained from an experiment in which the tester received new/old stimulus BCI paradigms prior to performing a lumbar Tap Test, and to acquire post-LTT new stimulus EEG signal data and post-LTT old stimulus EEG signal data obtained from an experiment in which the tester received new/old stimulus BCI paradigms subsequent to performing a lumbar Tap Test;
A probabilistic prediction module, said probabilistic prediction module being configured to: input the pre-LTT new stimulation EEG signal data, the pre-LTT old stimulation EEG signal data, the post-LTT new stimulation EEG signal data, and the post-LTT old stimulation EEG signal data of the tester obtained by said data acquisition module into the iNPH prediction model obtained by the above-described method for constructing an iNPH prediction model based on the new and old stimulation BCI paradigm, obtaining the iNPH prediction results.

The present invention also discloses a brain-computer interface system for predicting iNPH that may include:
An EEG cap for being worn on the head of a tester to obtain EEG signals from said tester;
An EEG acquisition device configured to be coupled to the electrodes of said EEG cap to acquire EEG signals of said tester acquired by said EEG cap;
A computer comprising a communication module, a memory, a processor, and a computer program stored on said memory and runnable on said processor, wherein said computer is configured to communicate with said electroencephalographic acquisition device to obtain an electroencephalographic signal from said tester, and wherein said processor is configured to implement the following steps in executing said computer program:

Obtain pre-LTT new stimulus EEG signal data and pre-LTT old stimulus EEG signal data obtained from the BCI paradigm experiment in which the tester received new/old stimuli prior to performing a lumbar Tap Test, as well as obtain post-LTT new stimulus EEG signal data and post-LTT old stimulus EEG signal data obtained from the BCI paradigm experiment in which the tester received new/old stimuli after performing a lumbar Tap Test;
Preprocessing was performed on the pre-LTT new stimulus EEG signal data, the pre-LTT old stimulus EEG signal data, the post-LTT new stimulus EEG signal data, and the post-LTT old stimulus EEG signal data;
The preprocessed pre-LTT new stimulation EEG signal data, the pre-LTT old stimulation EEG signal data, the post-LTT new stimulation EEG signal data, and the post-LTT old stimulation EEG signal data are inputted into the iNPH prediction model obtained by the above-described method for constructing an iNPH prediction model based on the new and old stimulation BCI paradigm, and a prediction result as to whether or not said tester is a patient with iNPH is obtained;
wherein said computer program includes an EEG analysis program for pre-processing the EEG signals acquired by said EEG acquisition device.

FIG. 9 exemplifies a schematic diagram of a physical structure of an electronic device, as shown in FIG. 9, which may include: a processor 810, a communications interface 820, a memory 830, and a communications bus 840, wherein the processor 810, the communications interface 820, the memory 830 accomplish communication with each other through the communication bus 840. The processor 810 may invoke logic instructions in the memory 830 to execute an iNPH prediction method based on the new/old stimulus BCI paradigms, the method comprising:

Obtain pre-LTT new stimulus EEG signal data and pre-LTT old stimulus EEG signal data obtained from the BCI paradigm experiment in which the tester received new/old stimuli before performing Lumbar Tap Test, as well as obtain post-LTT new stimulus EEG signal data and post-LTT old stimulus EEG signal data obtained from the BCI paradigm experiment in which the tester received new/old stimuli after performing Lumbar Tap Test;

The pre-LTT new stimulation EEG signal data, the pre-LTT old stimulation EEG signal data, the post-LTT new stimulation EEG signal data and the post-LTT old stimulation EEG signal data of the tester are inputted into the iNPH prediction model obtained as described above, and a prediction result of whether or not said tester is an iNPH patient is obtained;

Wherein said tester is a patient with suspected iNPH or a patient to be ruled out with iNPH, preferably said tester has a triad of manifestations of gait disturbance, urinary incontinence and cognitive impairment.

Furthermore, the logical instructions in the above-described memory 830 may be stored in a computer-readable storage medium when they can be realized in the form of a software function unit and sold or used as a separate product. Based on this understanding, the technical solution of the present invention essentially or contributing to the prior art or part of the technical solution may be embodied in the form of a software product that is stored in a storage medium comprising a number of instructions to cause a computer device (which may be a personal computer, a server, or a network device, etc.) to perform all or part of the steps of the method described in the various embodiments of the present invention. the method described in various embodiments of the present invention. The aforementioned storage media include: USB flash drive, removable hard disk, read-only memory (ROM, Read-Only Memory), random access memory (RAM, Random Access Memory), diskette or CD-ROM, and other media that can store program code.

The above-described embodiments of the device are merely schematic, wherein the units described as illustrated as separated components may or may not be physically separated, and the components shown as units may or may not be physical units, i.e., they may be located in a single place or they may also be distributed to a plurality of network units. Some or all of these modules may be selected to fulfill the purpose of the embodiment scheme according to actual needs. It can be understood and implemented by a person of ordinary skill in the art without creative labor.

Through the above description of the embodiments, it is clear to those skilled in the art that the embodiments can be realized with the aid of software plus the necessary general hardware platform, and of course also through hardware. Based on this understanding, the above technical solutions which essentially or rather contribute to the prior art may be embodied in the form of a software product, which may be stored in a computer-readable storage medium such as a ROM/RAM, a disk, a CD-ROM, etc., and comprises a number of instructions to enable a computer device (which may be a personal computer, a server, or a network device, etc.) to execute the respective embodiments or parts of embodiments. embodiments or certain portions of embodiments.

Finally, it should be noted that the above embodiments are only used to illustrate the technical solutions of the present invention, not to limit them; although the present invention has been described in detail with reference to the foregoing embodiments, a person of ordinary skill in the art should understand that it is still possible to make modifications to the technical solutions described in the foregoing embodiments, or to make equivalent substitutions for some of the technical features therein; and such modifications or substitutions do not take the essence of the corresponding technical solutions out of the spirit and scope of the technical solutions of the various embodiments of the present invention. These modifications or substitutions do not cause the essence of the corresponding technical solutions to depart from the spirit and scope of the technical solutions of the embodiments of the present invention.

What is claimed is:

1. A method for constructing an iNPH prediction model based on the new/old stimulus BCI paradigm, the method comprises:
performing new/old stimulus BCI paradigm experiments on the target population before Lumbar Tap Test to obtain pre-LTT new stimulus EEG signal data and pre-LTT old stimulus EEG signal data for the target population;
performing the same new/old stimulus BCI paradigm experiments on the target population after Lumbar Tap Test to obtain post-LTT new stimulus EEG signal data and post-LTT old stimulus EEG signal data for the target population;
performing pre-processing on the pre-LTT new stimulus EEG signal data, pre-LTT old stimulus EEG signal data, post-LTT new stimulus EEG signal data and post-LTT old stimulus EEG signal data to obtain the pre-LTT new stimulus event-related potential features, the pre-LTT old stimulus event-related potential features, the post-LTT new stimulus event-related potential features and the post-LTT old stimulus event-related potential features, wherein the event-related potential features are the P600 amplitude features;
training iNPH prediction model to obtain the iNPH prediction model based on the pre-LTT new stimulus event-related potential features, the pre-LTT old stimulus event-related potential features, the post-LTT new stimulus event-related potential features, and the post-LTT old stimulus event-related potential features; and
one of the target populations is iNPH patients.

2. The method for constructing the iNPH prediction model based on a new/old stimulus BCI paradigm according to claim 1, wherein said experiments with the new/old stimulus BCI paradigm on a target population comprise:
configuring EEG caps for the target population;
presenting a number of images to a target population at predetermined time intervals, receiving image response information from the target population, wherein, in the new/old stimulus BCI paradigm experiment, the first presentation of an image is defined as a new stimulus and the non-first presentation of an image is defined as an old stimulus, and the target population is asked to memorize the presented images, and the image response information includes response information in which the target population perceives that the image is first time appearing and response information in which the target population perceives that the image non-first appearance response information; and
while presenting an image to the target population and receiving image response information from the target population, EEG signal data of the target population is obtained by means of an EEG cap.

3. The method of constructing the iNPH prediction model based on the BCI paradigm for new/old stimuli according to claim 2, wherein said presenting a number of images to a target population according to a predetermined time interval comprises:
a number of rounds of image stimulation are administered to the target population, each round of image stimulation comprising a black-and-white image stimulation and a color image stimulation, the black-and-white image stimulation and the color image stimulation comprising a number of image stimulations, wherein there is a break between each round of image stimulation and between the black-and-white image stimulation and the color image stimulation, and wherein the black-and-white image and the color image are derived from different stimulation libraries; and
images are presented to the target population each time in accordance with a predetermined stimulus maintenance duration, wherein one-half of the images appear only once, another one-half of the images are repeated once and there is a stimulus interval between the first image presentation and the second image presentation.

4. The method for constructing the iNPH prediction model based on the BCI paradigm for new/old stimuli according to claim 2, wherein said EEG cap is configured for the target population, specifically:
the target population was equipped with EEG caps, which used standard Ag/AgCl electrodes, with electrodes placed with reference to the international 10-20 system, and the number of channels was 21 leads; and
the parameters of the EEG caps were set to a sampling rate of 1,000 Hz and a band-pass filtering of 0.1 to 200 Hz, and the acquisition process was performed with the top of the head of the target population as a reference, with the forehead grounded and the impedance between the scalp and the electrodes kept below 10KΩ.

5. The method for constructing the iNPH prediction model based on the new/old stimulus BCI paradigm according to claim 4, characterized in that said preprocessing comprises band-pass filtering, re-referencing, independent component analysis, data segmentation, and baseline correction; wherein
band-pass filtering is specified as: the filtering of EEG signal data by a band-pass filter with a filter range of 0.5 to 20 Hz;
re-referencing is specified as: the EEG signal data are re-referenced to the average of the left mastoid and right mastoid as a reference;
independent component analysis is specified as the identification of EOG and EMG components associated with artifacts in order to remove artifacts from the EEG signal data;
the data segmentation is specified as: defining the moment of appearance of the image stimulus as the zero moment, in the EEG signal data, the data is segmented and intercepted according to the first predetermined time window; and
the baseline calibration is specified as: using the EEG signal data segments of the second predetermined time window as a baseline, subtracting the EEG signal data segments of the first predetermined time window from the average value of the EEG signal data segments of the baseline to eliminate the drift of the EEG signal data relative to the baseline.

6. The method for constructing the iNPH prediction model based on the new/old stimulus BCI paradigm according to claim 5, characterized in that said pre-processing of the pre-LTT new stimulus EEG signal data, the pre-LTT old stimulus EEG signal data, the post-LTT new stimulus EEG signal data, and the post-LTT old stimulus EEG signal data, to obtain the pre-LTT new stimulus event-related potential features, the pre-LTT old stimulus event related potential features, post-LTT new stimulus event related potential features and post-LTT old stimulus event related potential features, including:
based on the preprocessed EEG signal data, the event-related potential features are obtained by averaging the EEG signal data of each trial separately at each electrode by overlaying the calculation.

7. The method of constructing the iNPH prediction model based on the new/old stimulus BCI paradigm according to claim 6, wherein the event-related potential features of the target population are expressed in the form of: a new stimulus event-related potential features before the lumbar Tap Test, an old stimulus event-related potential features before the lumbar Tap Test, a new stimulus event-related potential features after the lumbar Tap Test and an old stimulus event-related potential features after the lumbar Tap Test reflecting the iNPH The population did not have old-new effect before performing lumbar Tap Test, while old-new effect appeared after lumbar Tap Test;
where the old-new effect indicates that the P600 amplitude feature under the old stimulus has a more positive potential than the P600 amplitude feature under the new stimulus within a certain time frame;
said iNPH prediction model is trained to obtain an iNPH prediction model based on a pre-LTT new stimulus event-related potential feature, a pre-LTT old stimulus event-related potential feature, a post-LTT new stimulus event-related potential feature, and a post-LTT old stimulus event-related potential feature, as follows:
the iNPH prediction model is trained to obtain the iNPH prediction model based on the feature changes reflected in the pre-LTT new stimulus event-related potential features, pre-LTT old stimulus event-related potential features, post-LTT new stimulus event-related potential features, and post-LTT old stimulus event-related potential features of the target population.

8. A device for constructing an iNPH prediction model based on a new/old stimulus BCI paradigm, wherein the device comprises:
a data receiving module, said data receiving module being configured to: receive pre-LTT new stimulation EEG signal data, pre-LTT old stimulation EEG signal data, post-LTT new stimulation EEG signal data, and post-LTT old stimulation EEG signal data of the target population, wherein said pre-LTT new stimulation EEG signal data and pre-LTT old stimulation EEG signal data, and post-LTT new stimulation EEG signal data and post-LTT old stimulated EEG signal data, and post-LTT new stimulated EEG signal data and post-LTT old stimulated EEG signal data are obtained through the BCI paradigm experiments in which the target population received new/old stimulated BCI paradigm experiments before and after lumbar Tap Test, respectively;
a data processing module, said data processing module being configured to pre-process the pre-LTT new stimulus EEG signal data, the pre-LTT old stimulus EEG signal data, the post-LTT new stimulus EEG signal data and the post-LTT old stimulus EEG signal data received by said data receiving module, to obtain the pre-LTT new stimulus event-related potential feature, the pre-LTT old stimulus event-related potential feature, the post-LTT new stimulus event-related potential features, old stimulus event-related potential features, new stimulus event-related potential features, new stimulus event-related potential features and old stimulus event-related potential features after lumbar Tap Test, of which, the event-related potential features are P600 amplitude features;
a model training module, said model training module being configured to train to obtain an iNPH prediction model based on the pre-LTT new stimulus event related potential features, the pre-LTT old stimulus event related potential features, the post-LTT new stimulus event related potential features and the post-LTT old stimulus event related potential features obtained by said data processing module.

9. The iNPH prediction device based on a new/old stimulus BCI paradigm, it wherein the device comprises:
a data acquisition module, said data acquisition module being configured to acquire pre-LTT new stimulus EEG signal data and pre-LTT old stimulus EEG signal data obtained from an experiment in which the tester received new/old stimulus BCI paradigms prior to performing a lumbar Tap Test, and to acquire post-LTT new stimulus EEG signal data and post-LTT old stimulus EEG signal data obtained from an experiment in which the tester received new/old stimulus BCI paradigms after performing a lumbar Tap Test Data;
a probabilistic prediction module, said probabilistic prediction module being configured to input the pre-LTT new stimulus EEG signal data, the pre-LTT old stimulus EEG signal data, the post-LTT new stimulus EEG signal data, and the post-LTT old stimulus EEG signal data of the tester obtained by said data acquisition module into the method of constructing an iNPH prediction model based on the new/old stimulus BCI paradigm as described in claim 1 to obtain the iNPH prediction model of claim 7 to obtain the iNPH prediction results.

10. A brain-computer interface system for predicting iNPH, it wherein the system comprises:
an EEG cap for being worn on the head of a tester to obtain EEG signals from said tester;
an EEG acquisition device configured to be coupled to the electrodes of said EEG cap to acquire EEG signals of said tester acquired by said EEG cap;
a computer comprising a communication module, a memory, a processor, and a computer program stored on said memory and runnable on said processor, wherein said computer is configured to communicate with said electroencephalographic acquisition device to obtain an electroencephalographic signal from said tester, and wherein said processor is configured to implement the following steps in executing said computer program:
obtain pre-LTT new stimulus EEG signal data and pre-LTT old stimulus EEG signal data obtained from the BCI paradigm experiment in which the tester received new/old stimuli before performing Lumbar Tap Test, as well as obtain post-LTT new stimulus EEG signal data and post-LTT old stimulus EEG signal data obtained from the BCI paradigm experiment in which the tester received new/old stimuli after performing Lumbar Tap Test;
pre-processing is performed on the pre-LTT new stimulation EEG signal data, the pre-LTT old stimulation EEG signal data, the post-LTT new stimulation EEG signal data, and the post-LTT old stimulation EEG signal data;
inputting the preprocessed pre-LTT new stimulus EEG signal data, the pre-LTT old stimulus EEG signal data, the post-LTT new stimulus EEG signal data, and the post-LTT old stimulus EEG signal data into an iNPH prediction model obtained according to the method of constructing an iNPH prediction model based on the new/old stimulus BCI paradigm according to claim 1, and obtaining whether or not said tester is an iNPH patient is predicted;
wherein said computer program includes an EEG analysis program for pre-processing the EEG signals acquired by said EEG acquisition device.
